(19) United States
(12) Patent Application Publication
(10) Pub. No.: US 2013/0261672 A1
(43) Pub. Date: Oct. 3, 2013

(54) UNIVERSAL DISTRACTION DEVICE FOR BONE REGENERATION

(71) Applicant: Domonkos Horvath, Jestetten (DE)

(72) Inventor: Domonkos Horvath, Jestetten (DE)

(73) Assignee: CELGEN AG, Zug (CH)

(21) Appl. No.: 13/992,349

(22) PCT Filed: Dec. 7, 2011
(86) PCT No.: PCT/EP11/06133

§ 371 Date: Jun. 7, 2013

(30) Foreign Application Priority Data
Dec. 10, 2010 (DE) 10 2010 055 431.6

(51) Int. Cl. A61B 17/66

(52) U.S. Cl. CPC A61B 17/663
USPC 606/282

(57) ABSTRACT

The present invention relates to a distraction device for bone regeneration, especially in the jaw region, to the use of the distraction device for callus distraction, and to methods for callus distraction.

The present invention relates to a distraction device for bone regeneration, especially in the jaw region, to the use of the distraction device for callus distraction, and to methods for callus distraction.

Bone losses are currently filled in with bone substitute material or with autogenous or allogenic bone.

From a biological view, an autologous spongiosa transplant is the best substitute material for a bone. However, such transplants are only available to a limited extent and exhibit a high resorption rate after transplantation.

The materials and techniques employed in the prior art frequently provide inadequate bone quality, so that implants, for example, are not rigidly anchored in the beds thereof. Additionally, the bone substitute is frequently not sufficiently vascularized, and as a result the risk of infection is increased. Methods according to the prior art often also employ growth factors, which significantly increase the costs of the procedures.

Instead of using a bone substitute, lacking bone substance can also be partially filled in by way of bone regeneration. Segmental osseous discontinuity on long bones can thus be treated by way of distraction osteogenesis.

Callus distraction has been known for more than one hundred years. The most important biological stimulus for osteogenesis is mechanical stress. Piezoelectrical forces are released in the process, which activate osteoblasts and osteoclasts. Distraction osteogenesis induces new bone formation by triggering biological growth stimuli by slowly separating bone segments. This method achieves the direct formation of woven bone by way of distraction. Defined tensile stress during bone generation is essential. If such defined tensile stress is applied to bone fragments, the mesenchymal tissues in the gap and on the adjoining fragment ends show osteogenetic capacity. If sufficient vascular potency exists, progressive distraction results in metaplasia of the organized hematoma, also referred to as a blood clot, in a zone of longitudinally arranged, fibrous tissue, which under optimal external and internal conditions can convert directly into woven bone. However, an aggravating factor is that the bone tissue is subject to highly complex control during regeneration thereof.

WO 01/91663 A1 and U.S. Pat. No. 5,980,252 describe devices and methods for callus distraction by way of artificial interfaces, for example membranes. The membranes used there are flat plates or flat small plates, which are usually made of metal, for example titanium. The membranes are moved by way of a variety of devices and actuating elements such as screws or cable controls. Some of these actuating elements are very complex, or the lifting of the membrane can only be insufficiently adjusted. In addition, the actuating element often has to be anchored in the bone so as to move the membrane by way of this element. In the prior art, the planar, which is to say flat, membranes are seated on a bone defect and are moved perpendicular to the bone defect, away from the same.

The technical problem underlying the present invention is to provide means and methods for bone distraction that make it possible to carry out bone regeneration methods, especially in the jaw region, which overcome the drawbacks of the prior art.

The underlying technical problem of the present invention is also to provide distraction devices that allow for a simple and safe design.

The underlying technical problem of the present invention is also to provide distraction devices that allow exact adjustment of the movement of a distraction membrane.

The underlying technical problem of the present invention is also to provide distraction devices that allow for use in differing bone defects, especially in the jaw region, despite ready-made components.

The technical problem underlying the present invention is also to provide distraction devices, to use the same, and to provide methods that make it possible to regenerate non-planar bone sections, or bone sections located obliquely relative to the distraction direction, such as an alveolar ridge.

The present invention solves the underlying technical problem in particular by providing distraction devices, methods and uses according to the claims.

The present invention solves the underlying technical problem in particular by providing a distraction device for bone regeneration, comprising a distraction membrane and an actuating element actuating the distraction membrane, wherein the actuating element is designed as a toothed rack.

The present teaching includes in particular membranes, distraction devices and methods for bone regeneration, wherein preferably bones in the jaw region and/or in the periodontal region are to be regenerated.

In the present invention, the term ‘bone regeneration’ in particular is understood to also mean the regeneration of bone defects, for example after cystectomy, tumor surgery or trauma surgery or the like, regardless of the topography, and/or in particular also the regeneration of smaller bone defects caused by periodontitis, for example.

A distraction device for bone regeneration is preferred that comprises a distraction membrane and an actuating element actuating the distraction membrane, wherein the actuating element is designed as a toothed rack, wherein the distraction device comprises a gear system for moving the toothed rack, and wherein the gear system can be disposed in a stationary manner in the region of the bone to be regenerated in the oral cavity, and wherein the gear system comprises a housing having a passage and a threaded body, wherein the toothed rack is inserted in the longitudinal extension through the passage substantially without play, preferably without play, through the housing, and wherein the threaded body is rotatably mounted in the housing so that the threaded body and the toothed rack are operatively engaged with each other.

In a preferred embodiment, the toothed rack is curved, and in particular moderately or significantly curved. For example, the toothed rack can have a radius of 10 mm to 70 mm, such as in particular 15 mm or 60 mm. In an alternative embodiment, the toothed rack is not curved.

In a preferred embodiment, the cross-section of the tooth rack is not circular. In a preferred embodiment, the cross-section of the tooth rack is not rotationally symmetrical.

In a preferred embodiment, the cross-section of the tooth rack is polygonal, and in particular quadrangular or oval. In the case of a polygonal cross-section; the corners can also be rounded or flattened.

A cross-section of the toothed rack that is not circular or rotationally symmetrical has the advantage that the toothed rack can be secured or held by a device in a simple manner, in particular can be seated in a housing of a gear system, so that the toothed rack cannot rotate about the own axis thereof, and lateral displacement or rotation of the membrane can thereby be prevented, although the membrane is only guided by a toothed rack. The membrane can thus be prevented from rotating about its own axis, using only one actuating element, and thus lateral displacement or rotation of the membrane can be prevented, without requiring the use of several actuating elements. Advantageously, only one actuating element thus penetrates the mucous membrane.

In a preferred embodiment, the distraction membrane is secured to one end of the toothed rack. In a preferred embodiment, the counter-surface of the distraction membrane is secured to one end of the toothed rack. The toothed rack can be secured in the center, or approximately in the center, of the counter-surface of the membrane. However, the toothed rack can also be secured in any other location of the counter-surface of the membrane. This allows the membrane and the distraction device to be positioned well on a bone defect during use.

The membrane can be fixed to the toothed rack or secured so as to be able to rotate about the longitudinal axis of the toothed rack. In particular, the membrane can be secured to the toothed rack so as to be able to rotate about the longitudinal axis of the toothed rack.

For example, the membrane can be connected to the toothed rack by way of a rotatable element, so that the membrane can be rotated about the longitudinal axis of the toothed rack. To this end, the membrane can be designed so as to latch into the rotatable element in particular rotational angle positions.

A membrane that can be rotated about the longitudinal axis of the toothed rack has the advantage that positional movement of the membrane around two rotational axes simultaneously, or in particular successively, is possible.

The membrane can be connected to the toothed rack at any desired angle. The angle between the surface of the membrane and the longitudinal axis of the toothed rack can thus be freely selected, for example 50 to 130 degrees, in particular approximately 90 degrees, most particularly 90 degrees.

In a preferred embodiment, the distraction membrane is arched at least in one direction.

In a preferred embodiment, the distraction membrane is a membrane, wherein the membrane comprises a contact surface and a counter-surface, and wherein at least one sub-region of the contact surface and at least one sub-region of the counter-surface are arched. The membrane is thus arched over at least a portion of the length or width of the membrane.

In a preferred embodiment, the distraction membrane is a membrane, wherein the membrane comprises a contact surface, a counter-surface and at least one lateral face, and wherein the edges between the contact surface and the at least one lateral face and/or between the counter-surface and the at least one lateral face are rounded.

A preferred embodiment is a membrane, also referred to as a distraction membrane, that is suitable for callus distraction, especially in the jaw region, wherein the membrane is arched, and wherein the membrane comprises a contact surface, a counter-surface and at least one lateral face, and wherein the edges between the contact surface and the at least one lateral face and/or between the counter-surface and the at least one lateral face are rounded.

In a preferred embodiment, the distraction membrane is a membrane, wherein the membrane comprises a contact surface, a counter-surface and at least one lateral face, and wherein the at least one lateral face is bent, in particular bent toward the contact surface.

A preferred embodiment is a membrane that is suitable for callus distraction, especially in the jaw region, wherein the membrane is arched, and wherein the membrane comprises a contact surface, a counter-surface and at least one lateral face, and wherein the at least one lateral face is bent, in particular bent toward the contact surface.

In the context of the present invention, a membrane is understood to mean a medical membrane, which is suitable for the distraction of a bone, preferably of a jaw bone, especially in the dental field. Such a membrane is also referred to as a distraction membrane.

The present teaching includes in particular membranes, distraction devices and methods for bone regeneration, wherein preferably bones in the jaw region and/or in the periodontal region are to be regenerated.

In the present invention, the term ‘bone regeneration’ in particular is understood to also mean the regeneration of bone defects, for example after cystectomy, tumor surgery or trauma surgery or the like, regardless of the topography, and/or in particular also the regeneration of smaller bone defects caused by periodontitis, for example.

In the context of the present invention, a membrane is understood to mean a body that is plate-shaped, which is to say planar or flat, in the non-arched state. The membrane has a contact surface, which is used for osteoblasts to line or adhere to in the region of a bone defect, and a counter-surface located opposite the contact surface. These two surfaces can take on any shape, for example they can be round, oval, quadrangular or polygonal. The contact surface and the counter-surface of the membrane are preferably rectangular in the non-arched state. In the non-arched state, the sizes of these two surfaces in a rectangular membrane result from the length and width of the membrane. The membrane additionally has at least one lateral face, and more particularly four lateral faces if it is a rectangular membrane. In the non-arched state, the sizes of two of the lateral faces result from the height and the length of a rectangular membrane, and the sizes of the two remaining lateral faces result from the height and width of the membrane. According to the invention, the membrane is as thin as possible, which means that the sizes of the lateral faces are several times smaller than the size of the contact surface, and in the case of a quadrangular membrane, the height of the membrane is several times smaller than the length and the width of the membrane.

A distraction membrane, which is suitable for bone distraction in the jaw region, is preferred, wherein the membrane comprises a contact surface and a counter-surface, and wherein at least a sub-region of the contact surface and a sub-region of the counter-surface are arched, and wherein the membrane has rounded edges.

A membrane that comprises a contact surface, a counter-surface and at least one lateral face is preferred, wherein at least one sub-region of the contact surface and a sub-region of the counter-surface are arched, and wherein the edges between the contact surface and the at least one lateral surface and/or between the counter-surface and the at least one lateral face are rounded.

In a preferred embodiment, the entire contact surface and the entire counter-surface are arched.

Arching in the context of the present invention shall be understood to mean a curvature of surfaces, in the present invention the contact surface and the counter-surface. According to the invention, the membrane is preferably singly arched, which in the case of a rectangular membrane means that two mutually opposing lateral faces are curved and the two other lateral faces are not curved.

In one embodiment, the membrane is designed as a shell, which is to say a membrane that is singly or doubly curved or arched.

In one embodiment, the membrane is arched so that it has the shape of a segment of a spherical shell, for example a hemispherical shell. In a further embodiment, the membrane is arched so that it has the shape of a cylindrical shell.

In a preferred embodiment, the radius of the arching corresponds to the radius of a bone to be treated, for example a long bone or a cranial bone.

In a preferred embodiment, the radius of the arching corresponds to the radius of a jaw alveolar ridge to be treated.

In a preferred embodiment, the arching has a radius of at least 5 mm. In a preferred embodiment, the arching has a radius of no more than 15 mm. In a preferred embodiment, the arching has a radius of at least 5 mm and of no more than 15 mm.

In an alternative embodiment of the invention, the respective edges that are formed by two lateral faces can be rounded.

In a preferred embodiment, the membrane has rounded edges.

A membrane that comprises a rectangular contact surface, a counter-surface and four rectangular lateral faces is preferred, wherein the contact surface and the counter-surface are arched, and wherein all the edges of the membrane are rounded.

In a likewise preferred alternative embodiment, the membrane is shaped and dimensioned so as to cover at least a portion of the surface of a jaw bone facing the teeth. A planar or an arched membrane can thus be provided, which in the planar state, which is to say in the flat state, is bent approximately in a horseshoe shape and has a length so that the membrane can cover at least a sub-region of an alveolar ridge. It is possible in particular for the membrane to cover the majority, for example up to 80%, of an alveolar ridge, or an entire alveolar ridge. A person skilled in the art, for example a dental technician, will be readily able to determine the size and shape of a membrane that is required to cover a desired alveolar ridge region. Membranes thus shaped can advantageously be used to treat wider bone defects, for example bone defects that cover several missing teeth, and even the entire alveolar ridge.

The shape and size of the membranes can be ready-made or individually adapted to the bone defected to be treated.

In a further embodiment, the membrane has at least one further arching, and more particularly several additional archings having smaller radii.

In a preferred embodiment, the membrane has bent edges. In a preferred embodiment, the membrane has at least two bent lateral faces.

In a preferred embodiment, the membrane has at least one perforation.

In a preferred embodiment, the membrane comprises titanium. In a preferred embodiment, the membrane is made of titanium. In a further embodiment, the membrane can also be made of a biodegradable material or comprise the same.

In a preferred embodiment, the membrane is sand-blasted. In a preferred embodiment, the contact surface of the membrane is sand-blasted.

In a preferred embodiment, the contact surface of the membrane is coated.

In a preferred embodiment, the edges of the membrane are covered with a non-woven fabric or a film.

The membrane according to the invention can be intended for multiple uses or for single use. The membrane is preferably intended for single use because this is common practice with medical membranes, and the adhesive power of the surface of the membrane decreases upon contact with body fluid. The membrane according to the invention can be intended for single use in particular if the membrane was individually produced for a particular bone defect and/or if the membrane comprises biodegradable constituents, which break down on use.

In a preferred embodiment, the membrane according to the invention comprises at least one securing element. The at least one securing element is used to secure the membrane to at least one actuating element, and in particular a toothed rack. A securing element can be a perforation, an eyelet or an anchor point, for example. In an alternative embodiment, the securing element is used to secure the toothed rack. To this end, the securing element can be a perforation or a securing point, for example, such as a welding point or soldering point.

In a preferred embodiment, the distraction device is based on the system of a worm gear, a toothed rack and a membrane, wherein the rotatory movement of a threaded body, in particular of a screw, is translated into a translatory movement of the toothed rack and of the membrane secured to the toothed rack. The threaded body, in particular the screw, preferably has a thread having a lead p=0.1 to 0.5 mm, in particular approximately 0.3 mm, and in particular 0.3 mm, and the toothed rack is accordingly toothed. For example, a lead of 0.3 mm of the threaded body results in a travel of 0.3 mm for each full revolution of the threaded body.

In a preferred embodiment, the toothed rack, the membrane and/or the housing are made of titanium or a titanium alloy, in particular the material TiAl4V. In a preferred embodiment, the threaded body is made of titanium or a titanium alloy or stainless steel.

The present invention also relates to a distraction device according to the invention for use for callus distraction, in particular for reconstructing a bone, and especially a jaw bone by way of distraction.

In a preferred embodiment, the distraction device comprises a gear system, and in particular a self-locking gear system, for moving the toothed rack. In a preferred embodiment, the gear system can be disposed in a stationary manner in the region of the bone to be regenerated, and more particularly in the oral cavity.

In a preferred embodiment, the gear system comprises a housing having a passage and a threaded body, wherein the toothed rack is inserted in the longitudinal extension through the passage at least substantially without play, and preferably without play, through the housing, and herein the threaded body is rotatably mounted in the housing so that the threaded body and the toothed rack are operatively engaged with each other.

In a preferred embodiment, the gear system can be disposed in a stationary manner in the region of the bone to be regenerated, and more particularly in the oral cavity, by way of a fixation device.

In a preferred embodiment, the gear system is secured to a fixation device. In a preferred embodiment, the gear system is secured to the fixation device so that the secured connection has several degrees of freedom, whereby individual alignment of the toothed rack with respect to the fixation device and/or a bone defect is possible.

In a preferred embodiment, the fixation device is associated with the distraction device. In a preferred embodiment, the fixation device is a bridge, at least one bracket or at least one implant. In a preferred embodiment, the gear system is laminated into the fixation device. In an alternative embodiment, the gear system is screwed onto the fixation device.

In a preferred embodiment, the distraction device is suitable for callus distraction in the jaw region.

In a preferred embodiment, the distraction device is suitable for bone regeneration in the jaw region.

In a preferred embodiment, the distraction device is suitable for periodontal regeneration in the jaw region.

In a preferred embodiment, the distraction device is intended for use in a medical procedure, and in particular in a surgical procedure.

In a preferred embodiment, the distraction device is intended for use during bone regeneration by way of distraction, in particular in the jaw region.

The distraction device according to the invention can be intended for multiple uses or for single use. The distraction device according to the invention can be intended for single use in particular if the distraction membrane was individually produced for a particular bone defect and/or if the membrane comprises biodegradable constituents, which break down on use. The distraction device according to the invention can also be intended for single use, in particular, if the device comprises a fixation device that was individually produced for the fixation with a particular bone defect. It is common practice to use bone screws and a medical membrane, in particular a distraction membrane, only once, even if these are made of non-biodegradable material, such as titanium, because not only do these often need to be individually adapted, but the surface is also modified by the one-time use, so that further use would often take place under less than ideal conditions.

In an alternative embodiment, the distraction device according to the invention is a distraction device comprising a membrane for periodontal regeneration. Periodontal regeneration shall be understood to mean regeneration of the periodontium, which is to say not only of the bone, but also of the periodontal ligament, the periodontal tissue, the gingiva and the papilla, for example by way of guided tissue regeneration (GTR). In a preferred embodiment, the membrane for periodontal regeneration has such small dimensions that the same can also be used in interdental spaces. In a preferred embodiment, the membrane for periodontal regeneration is very thin. In a preferred embodiment, the membrane for periodontal regeneration is shaped so that the membrane comprises at least one lobular extension or a segment that can be inserted into an interdental space. In a preferred embodiment, the membrane for periodontal regeneration is a single-piece, two-piece or multi-piece membrane. In a preferred embodiment, the membrane for periodontal regeneration comprises at least one securing element for securing a bone screw, for example at least one perforation.

The present invention also relates to the use of a device according to the invention in a medical procedure, in particular in a surgical procedure.

The present invention further relates to the use of the distraction device according to the invention for callus distraction, especially in the jaw region.

The present invention also relates to a kit, including a membrane according to the invention and a toothed rack according to the invention.

The present invention also relates to a kit, including several membranes according to the invention and several toothed racks according to the invention.

The present invention also relates to a kit, including a medical membrane, in particular a distraction membrane, for bone regeneration, a toothed rack, a clasp, in particular a housing, and a threaded body, wherein the toothed rack can be inserted into the clasp, in particular the housing, and the threaded body can be mounted in the clasp, in particular the housing, so that the thread of the threaded body engages in the teeth of the toothed rack.

The kit preferably includes instructions for use. The instructions for use preferably include descriptions of how the kit can be used to carry out a callus distraction.

A preferred embodiment is a kit according to the invention for use in medical procedures, in particular surgical procedures, preferably during bone distraction, especially in the jaw region. A further preferred embodiment is the use of a kit according to the invention for the production of a device according to the invention.

The present invention also relates to a kit for use with bone distraction in the jaw region, including a distraction membrane for bone regeneration, a toothed rack, a housing, a threaded body, and instructions for use, wherein the toothed rack can be inserted into the housing, and the threaded body can be mounted in the housing so that a thread of the threaded body engages in teeth of the toothed rack.

The present invention also relates to methods for callus distraction, and more particularly for reconstructing a jaw bone by way of distraction, wherein a membrane of a distraction device according to the invention is applied to a bone segment to be regenerated and tensile stress is applied to this membrane by way of the toothed rack of the distraction device according to the invention. The membrane is thus moved away from the bone defect by the toothed rack at a particular rate. The rate is preferably approximately 0.1 mm to 2 mm per day, and more particularly 0.5 mm to 2 mm per day. However, the rate can also be approximately 1 mm per day. Without being bound to theory, in particular a distance of approximately 1.5 mm between the membrane and the bone is advantageous at the start of the procedure for such distraction methods.

Slowly moving the membrane away from the bone defect can take place continuously or discontinuously, for example daily or twice a day.

The invention will be described in greater detail hereafter based on the figures. In the drawings:

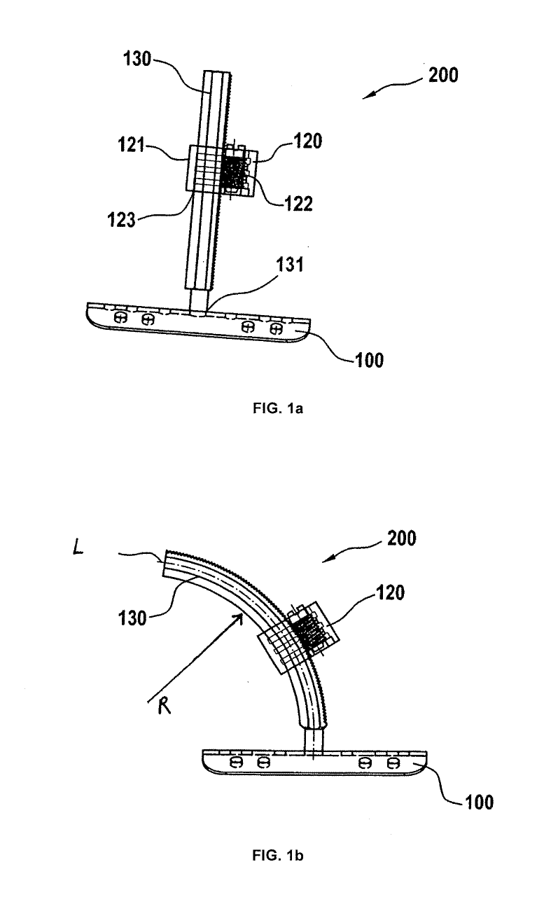

FIG. 1a shows a distraction device according to the invention comprising a straight toothed rack;

FIG. 1b shows a distraction device according to the invention comprising a curved toothed rack;

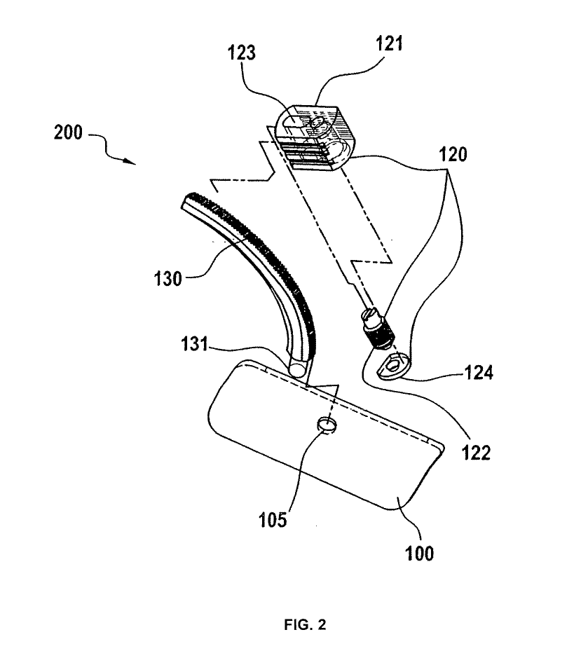

FIG. 2 is an exploded view of the distraction device of FIG. 1b;

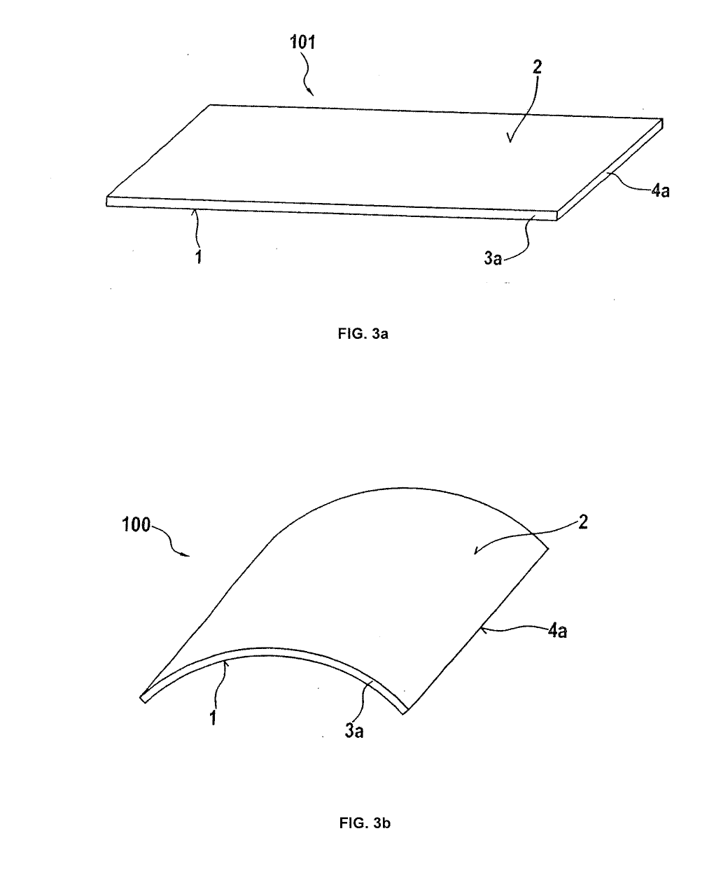

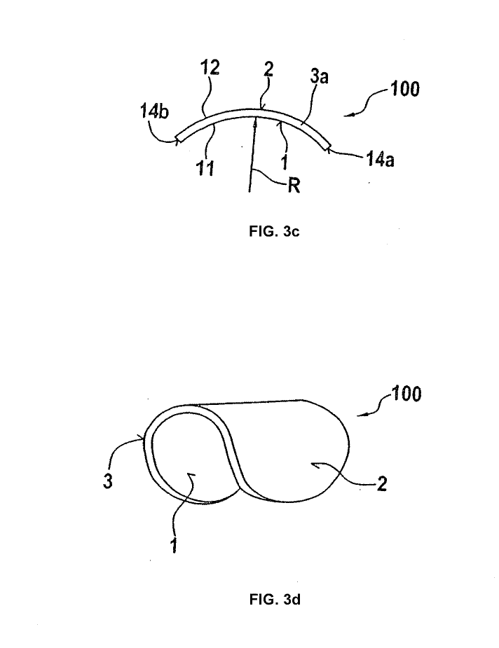

FIG. 3a shows a membrane of the prior art;

FIG. 3b shows a membrane according to the invention, comprising a rectangular contact surface;

FIG. 3c is a side view of the membrane according to FIG. 1 b;

FIG. 3d shows a membrane according to the invention, comprising a circular contact surface;

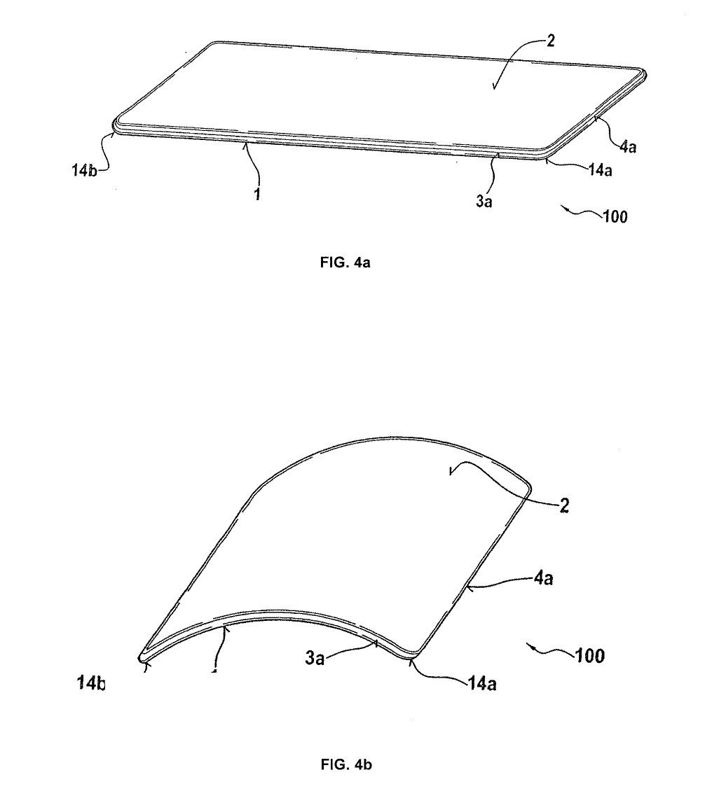

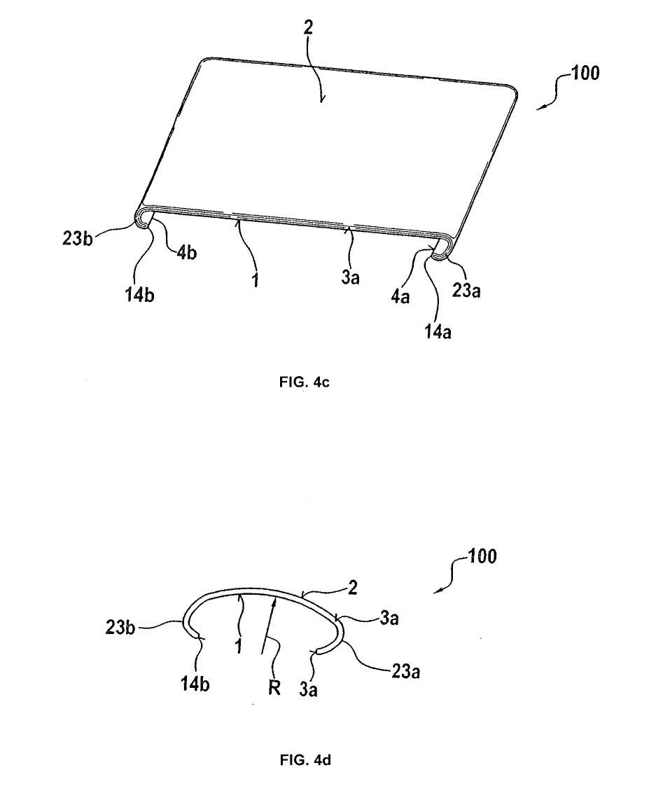

FIG. 4a shows a membrane according to the invention, comprising rounded edges;

FIG. 4b shows an arched membrane having rounded edges;

FIG. 4c shows a membrane having bent edge regions;

FIG. 4d shows an arched membrane having bent edge regions and rounded edges;

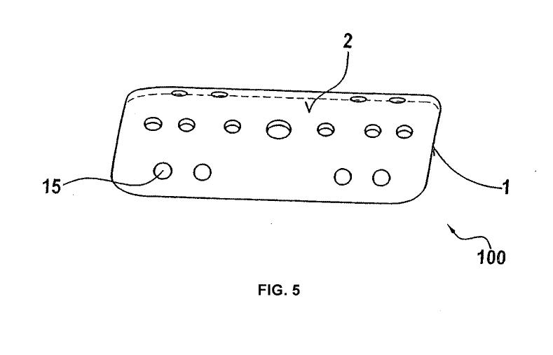

FIG. 5 shows an arched membrane having perforations;

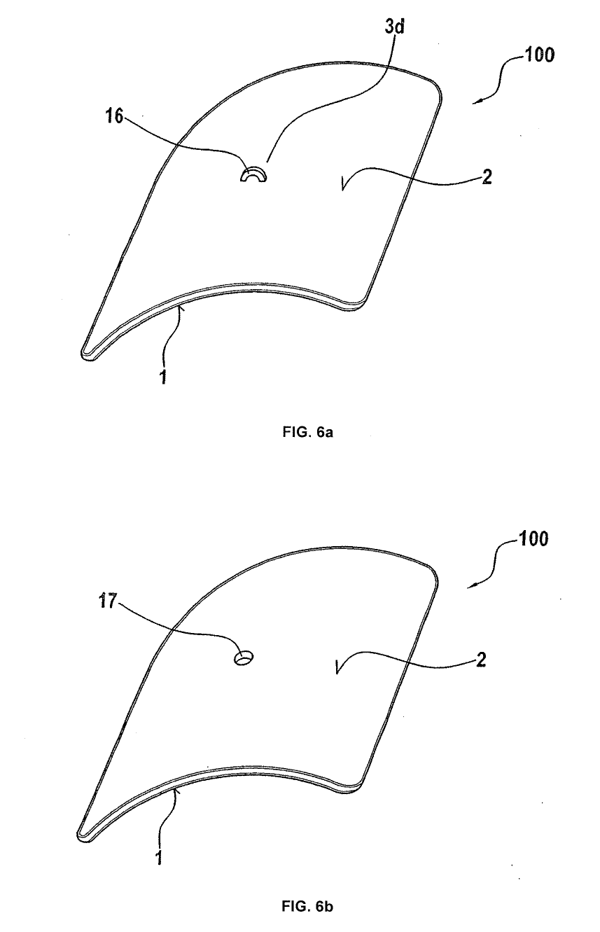

FIG. 6a shows a membrane comprising an eyelet as a securing element;

FIG. 6b shows a membrane comprising a hole as a securing element;

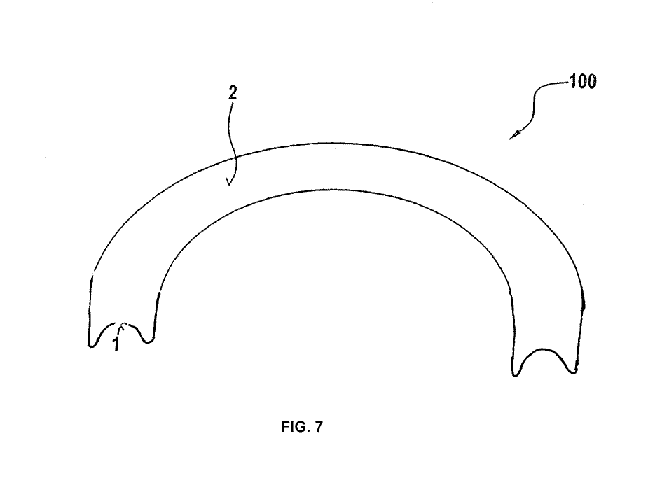

FIG. 7 shows a horseshoe-shaped membrane for treating large-surface-area jaw defects;

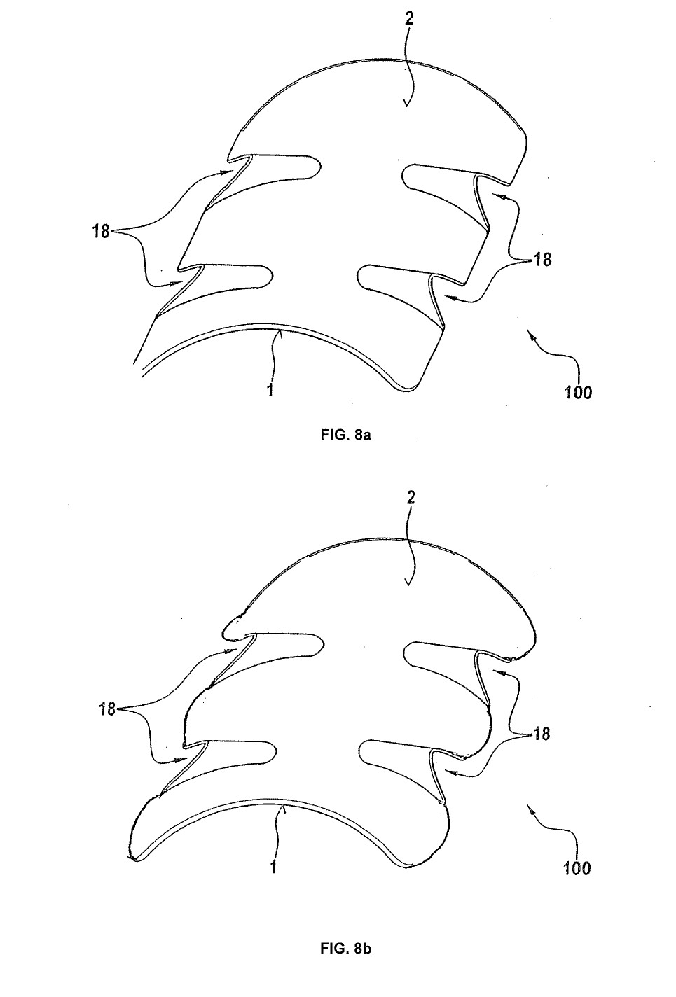

FIG. 8a shows a membrane comprising further archings for interdental papilla;

FIG. 8b is an alternative embodiment of the membrane comprising further archings for interdental papilla;

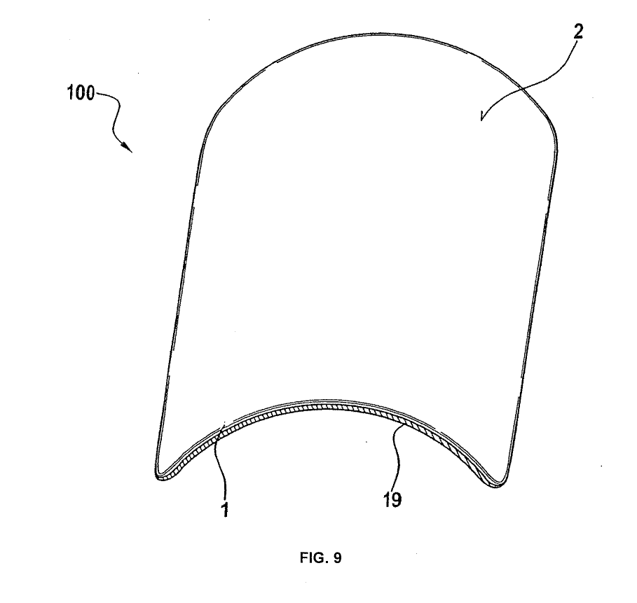

FIG. 9 shows a membrane, the contact surface of which is coated; and

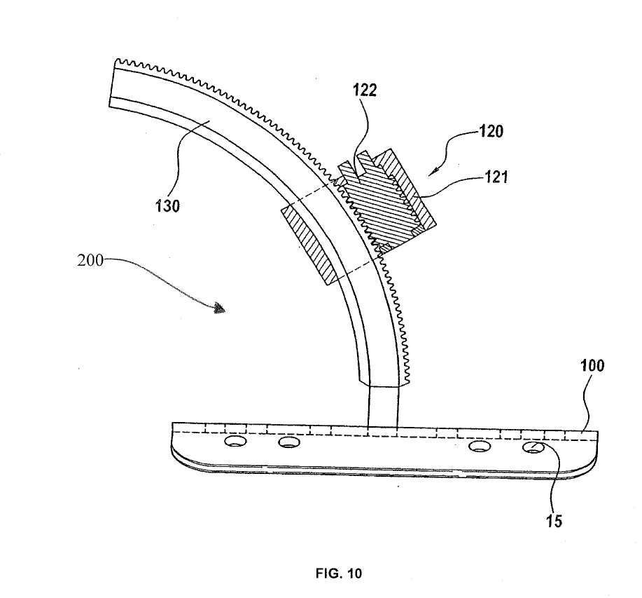

FIG. 10 is a further illustration of a distraction device, comprising a membrane, a fixation device and an actuating element in the form of a toothed rack, which connects the fixation device to the membrane.

FIG. 1a shows a distraction device 200 comprising a membrane 100. The membrane 100 can be arched or planar. Preferred membranes have been disclosed in the description. The membrane is connected to a gear system 120 by way of an actuating element designed as a straight, which is to say non-curved, toothed rack 130. The gear system 120 is preferably a self-locking gear system. The gear system 120 comprises a housing 121 having a passage 123 and a threaded body 122. The housing 121 comprising the passage 123 can take on the shape of a clasp. The toothed rack 130 is inserted in the longitudinal extension through the passage 123 at least substantially without play through the housing 121. Play can be eliminated, for example, by designing the inner sides of the passage 123 that come in contact with the toothed rack 130 as sliding bearings. The threaded body 122 is rotatably mounted in the housing 121 so that the threaded body 122 and the toothed rack 130 are operatively engaged with each other. A person skilled in the art will know suitable shapes for the thread of the threaded body 122 and for the teeth of the toothed rack 130 so as to allow good engagement of the thread in the teeth.

In a preferred embodiment, the threaded body 122 can be a screw. In an alternative embodiment, the threaded body 122 can be a spindle.

The membrane 100 is preferably connected at one end 131 of the toothed rack 130 to this end 131 of the toothed rack 130 by way of bonding and/or friction fit. Preferably the counter-surface 102 of the membrane 100 is connected to the toothed rack 130, but in any case not the contact surface 101. For example, the membrane 100 can be welded to the toothed rack 130, and in particular welded thereto by way of laser welding, soldered thereto or glued thereto. The membrane 100 and toothed rack 130 can also be designed integrally with each other. As an alternative, the membrane 100 can also comprise a hole through which a sub-section of the toothed rack 130, which can be pin-shaped for example, is inserted, whereby the membrane 100 is secured to the toothed rack 130. The hole of the membrane 100 can also comprise an internal thread, and the end section of the toothed rack 130 located at the end 131 can comprise a matching external thread, so that the membrane 100 is screwed onto the toothed rack 130.

In the figure, the membrane 100 is secured to the toothed rack 130 at an angle of 90 degrees. However, the same can also be secured at any other arbitrary angle. This allows the membrane 100 to be adjusted in the oral cavity, so that the membrane 100 can be seated against the bone defect with a precise fit and moved away from the bone defect at a desired inclination during distraction. This allows vertical parallel distraction to be carried out in the transverse direction as well, in accordance with the angle. In an alternative embodiment, the membrane 100 is secured to the toothed rack 130 at an angle that is not 90 degrees, for example at an angle of 40 to 89.5 degrees or of 91 degrees to 140 degrees.

It is also possible for the membrane surface to be rotated about the axis of the toothed rack by any arbitrary angle. This allows a rectangular membrane, for example, to be adjusted so that it covers the bone defect to be treated over the entire area to as great an extent as possible.

When the threaded body 122 is turned in the housing 121 of the gear system 120, for example using a screwdriver, an Allen wrench or a regular wrench, the toothed rack 130 moves in the gear system, whereby the membrane 100 is pushed away from the gear system 120 or pulled toward the gear system 120 by way of the toothed rack 130.

The gear system 120 can be disposed in a stationary manner in the region of the bone to be regenerated, in particular in the oral cavity, by way of a fixation device. For example, it can be laminated into the fixation device. As a result, the toothed rack to be individually aligned. This can be done using a spherically arched screw nut, for example. As an alternative, the gear system can also be secured by way of at least one screw, and in particular by way of one screw, in the fixation device. This can preferably be done in a way so as to provide the secured mechanical connection with several degrees of freedom, to assure that the individual alignment of the toothed rack is preserved. However, different options for securing the gear system 120 to the fixation device are also possible, which a person skilled in the art, for example a dental technician, will know.

The fixation device can be any device by way of which the distraction device 200 can be fixed to a tooth, or several teeth, or to the jaw bone, using the gear system 120. For example, the fixation device can be a bridge, in particular one that can be secured to the two teeth bounding the bone defect. However, the fixation device can also be a bracket, a bone screw, or a temporary implant.

FIG. 1b shows a distraction device 200 comprising a membrane 100. The membrane is connected to a gear system 120 by way of an actuating element designed as a curved toothed rack 130. The distraction device 200 differs from the distraction device of FIG. 1a only by the curved toothed rack 130. All preferred and alternative embodiments of FIG. 1a are thus also considered to be disclosed for FIG. 1b. The toothed rack 130 is curved along the longitudinal axis L thereof.

The radius of curvature R of the toothed rack 130 can be arbitrarily selected. The curvature of the toothed rack preferably has a radius R of at least 1 mm. The curvature of the toothed rack preferably has a radius R of no more than 150 mm. The curvature of the toothed rack preferably has a radius R of at least 1 mm and no more than 150 mm, and more particularly of at least 10 mm and no more than 100 mm. Preferred radii R are approximately 15 mm and approximately 60 mm, and more particularly 15 mm and 60 mm, for example. For example, the toothed rack 130 can be curved so that the teeth of the toothed rack 130, as shown in FIG. 1b, are preferably located on the convex surface of the curved toothed rack 130. However, alternatively the teeth of the toothed rack 130 can also be located on the concave surface of the curved toothed rack 130. However, the teeth can also be located on one of the side flanks of the curved toothed rack 130.

Compared to a non-curved toothed rack, or distraction devices from the prior art, the curved toothed rack advantageously allows bone sections that are not planar or located obliquely relative to the distraction direction, for example an alveolar ridge, to be regenerated. It is also possible to carry out a vertical parallel distraction in a transversal direction in accordance with the angle, in particular if the membrane is secured to the toothed rack at an angle of 90 degrees. A curved toothed rack can advantageously be used to straighten an alveolar ridge when the alveolar ridge is inclined and to widen the ridge in the transversal direction, especially with a toothed rack curvature having a radius of 5 mm to 25 mm.

FIG. 2 shows an exploded view of the distraction device 200 of FIG. 1b. Shown is the membrane 100, the curved toothed rack 130 and the gear system 120. The gear system 120 is composed of the housing 121 having a passage 123, the threaded body 122, and an optionally provided cover 124, which prevents the threaded body 122 from sliding out of the housing 121 after the threaded body 122 has been introduced into the housing 121 and forms part of the housing 121 when the gear system 120 is assembled.

A section of the toothed rack 130 located at the end 131 of the toothed rack 130 has a pin shape, so that the membrane 100 can be pushed onto the toothed rack 130 via a hole 105 and secured there.

FIG. 3a shows a distraction membrane 101 from the prior art. The distraction membrane 101 has a contact surface 1, which is not visible here, and a counter-surface 2, and of the four lateral faces, the faces 3a and 4a, which adjoin each other, can be seen. Such a membrane can be used as described in WO 01/91663 A1 and U.S. Pat. No. 5,980,252, wherein the contact surface 1 faces a bone and the membrane is moved away, for example pulled away, from the bone at a particular rate, for example approximately 1 mm per day, using a distraction device.

FIG. 3b shows a membrane according to the invention that is arched. The membrane 100 has a contact surface 1 and a counter-surface 2. In addition, the membrane has four lateral faces 3a, 3b, 4a and 4b, of which only the two mutually adjoining lateral faces 3a and 4a can be seen. In a preferred embodiment, the membrane is singly arched, as shown in FIG. 1b. According to the invention, the contact surface 1 is concavely curved and the counter-surface 2 is convexly curved. With a single curvature of the shown membrane 100 having rectangular faces, two mutually opposing lateral faces 3a and 3b are curved and the other two mutually opposing lateral faces 4a and 4b are not curved.

The arched geometry of the membrane advantageously results in greater stability of the membrane against warping. This makes it possible to use distraction membranes having a very small membrane height, which is to say membrane thickness. This is advantageous when using such a membrane for callus distraction in the jaw region because here the membrane is placed under the mucous membrane, and membranes having a large height result in tension in the mucosal flaps, which can cause ischemia associated with tissue necroses. This can also result in membrane exposure, as the result of which a membrane that is subject to bacterial colonization has to be removed. The arched geometry of a membrane according to the invention now allows stable membranes having a low height to be used, so that tension on the mucous membrane can be avoided.

The membrane preferably has a height of no more than 1 mm, and more particularly 0.5 mm.

In a preferred embodiment, the membrane 100 has a length of at least 5 mm and no more than 120 mm and a width of at least 5 mm and no more than 120 mm. For example, the membrane can have a length of approximately 20 mm and a width of approximately 10 mm. The length and width information applies to the membrane in the non-arched state. The membrane can in particular have approximately the width of an alveolar ridge and the length of a portion of the alveolar ridge or of the entire alveolar ridge.

A wide variety of suitable materials for distraction membranes are known to a person skilled in the art. The membrane is preferably made of a biocompatible material. The membrane is preferably made of a metal, in particular titanium. Membranes made of metals such as titanium have the advantage that they are very stable, despite having a small height.

However, alternatively, the membrane can also be made of a biocompatible plastic material. The plastic material is preferably a bioresorbable plastic material. These have the advantage that they do not have to be removed after the distraction.

FIG. 3c shows a side view of the arched membrane 100 according to the invention. Shown is the curved lateral face 3a, the edge 11 of which adjoins the concave contact surface 1 and the curved edge 12 of which adjoins the convex counter-surface 2. The edges 14a and 14b of the lateral face 3a adjoin the lateral faces 4a and 4b.

The membrane 100 is arched evenly over the entire contact surface 1. However, it is also possible that only sub-regions of the contact surface 1 are arched, for example only the center third of the edge 11 is arched. The radius R of the arching can also be different in various regions of the contact surface. According to the invention, the radius R of the arching of the contact surface 1 is preferably adapted to the natural shape of a jaw. The radius of the arching of the contact surface is preferably at least 5 mm and no more than 15 mm, particularly preferably at least 5 mm and no more than 12 mm, and in particular at least 6 mm and no more than 10 mm. The radius R of the arching of the contact surface 1 is preferably at least 5 mm, particularly preferably at least 6 mm. The radius R of the arching of the contact surface 1 is preferably no more than 15 mm, particularly preferably 12 mm, and in particular no more than 10 mm. The radius R of the arching of the contact surface 1 is preferably approximately 6 to 7 mm.

An arched membrane according to the invention thus not only has the advantage of increased stability with a low height, but such a membrane is advantageously also shaped so that the arching is consistent with the anatomical and physiological conditions of the bone to be regenerated. This allows the bone to be regenerated over the entire contact surface of the membrane because this membrane, during distraction, has approximately the same distance from the regenerating bone at every point.

FIG. 3d shows an arched membrane 100 according to the invention, in which the contact surface 1 and the counter-surface 2 are not rectangular, but circular. Such a membrane thus has only one lateral face 3.

FIG. 4a shows a planar membrane 100 according to the invention, having rounded edges. A membrane on which all edges are rounded is preferred. However, it is also possible that only the edges between the contact surface 1 and the at least one lateral face 3a, 4a and/or the edges between the counter-surface 2 and the at least one lateral face 3a, 4a are rounded. Because the lateral faces 3a, 4a are very narrow due to the low height of the membrane, it is also possible for the edges between the contact surface 1 of the membrane 100 and the at least one lateral face 3a, 4a of the membrane, and between the counter-surface 2 of the membrane 100 and the at least one lateral face 3a, 4a of the membrane 100, to be rounded so that the two rounded regions transition into each other. It is thus possible for the at least one lateral face 3a, 4a to be rounded. This is shown in FIG. 2b. The edges 14a, 14b between the individual lateral faces 3a, 4a are also preferably rounded. Preferably not only the edges, but also the corners of the membrane are rounded.

Rounding the edges of a membrane advantageously prevents the edges from injuring the surrounding tissue, for example by cutting or crushing the tissue or fine vessels and capillaries, when the membrane is moved during distraction. Because of the rounded edges and/or corners, a membrane according to the invention can advantageously slide past the adjacent tissue without damaging the same. The rounded edges advantageously make it easier to adapt the mucous membrane over the membrane.

FIG. 4b shows an arched membrane 100 having rounded edges 4a, 4b.

The combination of the arching according to the invention and rounding of the edges according to the invention advantageously results in a membrane that protects the adjacent tissue particularly well during the distraction, because the membrane edges do not compress the fine vessels and capillaries of the adjacent tissue, which are very important for providing tissue nutrients to the mucous membrane covering the membrane. This prevents premature membrane exposure.

According to a further embodiment, the edges can additionally be covered with a non-woven fabric or a film. The non-woven fabric or the film can be bioresorbable or non-bioresorbabie. Covering the edges with a non-woven fabric or a film offers additional protection for the adjacent tissue, in particular if the membrane is made of a very hard material, such as titanium for example.

FIG. 4c shows a planar membrane 100 having rounded edges 14a, 14b, wherein the lateral faces 4a, 4b are also bent toward the contact surface 1. The membrane is thus bent in at least two outer regions of the contact surface 1 and of the counter-surface 2, in particular it is bent toward the contact surface 1.

Bending the at least one lateral face 4a, 4b of the membrane also protects the adjacent tissue when the membrane is used because the tissue is seated against the bend, and not against a sharp edge. The edges therefore do not necessarily have to be rounded when the lateral faces are bent.

Because, during use, the membrane 100 is typically moved in the direction of the counter-surface 2, the lateral faces 4a, 4b are preferably bent in the direction of the contact surface 1.

FIG. 4d shows an arched membrane 100 having rounded edges 14a, 14b, wherein the lateral faces 4a, 4b are also bent toward the contact surface 1. The bends 23a, 23b of the edges 4a, 4b can be seen here as amplified arches of the basic arching of the membrane 100. In a preferred embodiment, the bends thus have a radius that is smaller than the radius R of the basic arching of the membrane.

FIG. 5 shows an embodiment of an arched membrane 100, wherein the membrane 100 is perforated so that the contact surface 1 and the counter-surface 2 are connected to each other by at least one hole 15, and more particularly by a plurality of holes, which can be distributed in particular over the entirety of the surfaces. In a preferred embodiment, the perforation holes have a diameter at least approximately 0.3 mm and no more than approximately 1.3 mm.

Perforation holes, and perforation holes having a diameter of approximately 1 mm in particular, allow capillaries to grow through the membrane, whereby excellent blood circulation and immune defense are ensured in the region of the newly formed bone. The perforations allow good blood flow through the mucous membrane covering the membrane and through the regenerated tissue between the membrane and the bone.

The number of perforation holes preferably varies depending on the size of the membrane. For example, a membrane having a length of approximately 20 mm and a width of approximately 10 mm can have approximately 10 to 20 perforation holes. Such a ratio of the number of perforation holes to the membrane surface provides an optimum between the total hole surface promoting blood circulation and the adhesion surface for osteoblasts adhering to the membrane during the distraction process.

FIG. 6a shows a membrane 100 that is arched according to the invention, comprising a eyelet 16 as a securing element. The eyelet 16 is located on the counter-side [sic: counter-surface] 2 of the membrane 100. A distraction device can also be attached to the eyelet 16, for example by way of a wire. By virtue of the distraction device, the membrane 100 can be used for callus distraction in the defect region of a bone by being pulled away from a bone defect at an adapted rate of approximately 1 mm per day.

FIG. 6b shows a membrane 100 that is arched according to the invention, comprising a hole 17 as a securing element. For example, a toothed rack of a distraction device can be inserted into the hole 17 and secured to the membrane 100, for example by way of welding, in particular laser welding, soldering or gluing.

FIG. 7 shows a preferred horseshoe-shaped embodiment of the membrane 100 for treating large-surface-area jaw bone defects.

The membrane 100 shown can be used, for example, if all the teeth of a jaw are missing and the jaw bone has to be regenerated so as to be able to perform implants. If not all the teeth of the jaw are be missing, but only a large number of teeth that are located next to each other, the membrane 100 can be shortened and adapted accordingly.

FIG. 8a shows an alternative embodiment of the membrane 100 according to the invention, in which additional archings 18 for the interdental papilla are provided. These additional archings are adapted to the shape of the jaw bone forming the interdental papilla. The jaw bone is raised between two adjacent teeth in a region of approximately 2 mm, whereby the periodontium is higher there than in the region of the teeth. The additional archings can be provided so as to preserve this raised bone area even after bone regeneration by way of distraction. In order for the archings to follow the shape of the interdental papilla, these preferably have a radius of 0.5 mm to 1.5 mm, in particular approximately 1 mm, and are located offset by an angle of approximately 90 degrees relative to the first arching according to the invention. In a planar membrane, these archings are located along the longitudinal sides of the membrane running parallel to the alveolar ridge. A person skilled in the art, for example a dental technician, will be able to determine without great effort the dimensioning and positioning of additional archings that follow the shape of interdental papilla.

FIG. 8b shows an alternative embodiment of the membrane 100 of FIG. 6a. In this embodiment, the membrane surfaces 1, 2 are extended downward further between the additional archings 18, so that the alveolar ridge can also be covered laterally by the membrane.

FIG. 9 shows a preferred embodiment of the membrane 100, in which the contact surface 1 of the membrane 100 is covered by a coating 19.

In a preferred embodiment, the contact surface 1 of the membrane 100 is coated with hydroxylapatite. In a further preferred embodiment, the contact surface 1 of the membrane 100 is coated with a bone substitute material, in particular a natural or synthetic bone substitute material.

The bone substitute material is preferably a natural bone substitute material, for example made of the mineral component of bones, in particular autogenous, allogenic or xenogenic bone, for example animal bones, and bovine bones in particular. A suitable bone substitute material is Bio-Oss®, which is available from Geistlich, for example:

In a preferred embodiment, the contact surface of the membrane is coated with a bone substitute material and a biodegradable glue, in particular a fibrin glue. The bone substitute material is preferably joined to the contact surface of the membrane by way of the fibrin glue. Such a coating advantageously allows the newly generated bone tissue to adhere to the membrane before and during the distraction, because the bone tissue can adhere well to the biological substitute material. In addition, such a coating allows for easy detachment, in particular even autonomous detachment of the membrane from the newly formed bone after completion of the distraction, because the fibrin glue is biodegradable, and thus is biologically degraded and decomposed during, and in particular after, the distraction. The coating made of bone substitute material thus detaches from the contact surface of the membrane. The membrane can be removed without having to be separated further from the bone, and the bone substitute material can remain in the restored bone defect.

FIG. 10 shows a distraction device 200, comprising an arched membrane 100 having perforations 15, a fixation device 120, and an actuating element in the form of a toothed rack 130 that connects the fixation device 120 to the membrane 100. The distraction membrane 100 is secured to one end of the toothed rack 130. The toothed rack 130 is curved, but can also be straight. The fixation device 120 comprises a gear system, in particular a self-locking gear system, for moving the toothed rack, wherein the gear system can be disposed in a stationary manner in the region of the bone to be regenerated, in particular in the oral cavity. The gear system comprises a housing 121 having a passage and a threaded body 122, wherein the toothed rack 130 is inserted in the longitudinal extension through the passage at least substantially without play through the housing 121, and wherein the threaded body 122 is rotatably mounted in the housing 121 so that the threaded body 122 and the toothed rack 130 are operatively engaged with each other. The gear system 120 can be disposed in a stationary manner in the region of a bone to be regenerated, in particular in the oral cavity, by way of additional elements of the fixation device 120, for example brackets or bridges.

Of course, the preferred embodiments shown in FIGS. 1 to 10 can be arbitrarily combined with each other.

The present invention will be described in greater detail based on the following example.

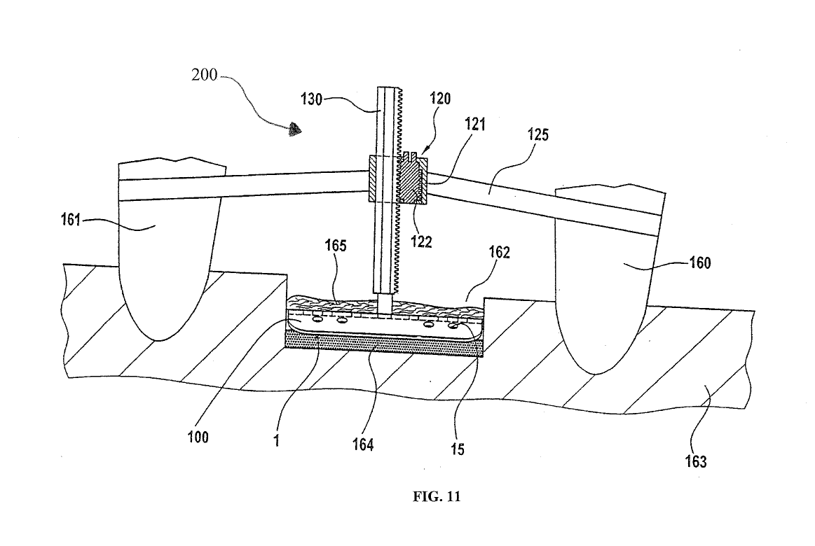

FIG. 11 shows a distraction device 200 comprising an arched membrane 100 according to the invention. The membrane is connected to a gear system 120 by way of a toothed rack 130.

The gear system 120 is laminated into a bridge 125, which is secured to two teeth 160, 161. The insertion, and more particularly the lamination, can be carried out individually, so that positional differences between the center of the alveolar ridge and the bridge can be compensated for, especially if the bridge is not located directly above the center of the alveolar ridge. A gap having a bone defect 162 of the arched jaw bone 163 is located between the teeth 160, 161. The contact surface 1 of the membrane 100 is seated against the jaw bone in the bone defect. Because the membrane 100 approximately follows the arching of the jaw bone 163, the contact surface 1 is seated thereon evenly. A coagel 164 has formed between the jaw bone 163 and the membrane 100, with osteoblasts also being present in this coagel. These osteoblasts can adhere to the contact surface 1 of the membrane 100. If the threaded body 122 in the housing 121 of the gear system 120 is now turned using a screwdriver or the like, the toothed rack 130, together with the membrane 100, moves upward. The threaded body 122 can be turned so that the membrane 100 moves away from the jaw bone 163 at a rate of approximately 0.5 to 2 mm, and more particularly approximately 1 mm, per day. For example, the threaded body 122 can be turned once a day by a particular revolution, or twice a day by half this revolution.

The rotatory movement of the threaded body 122 is thus translated into a translatory movement of the toothed rack 130 and of the membrane 100 secured to the toothed rack 130. In the present example, the thread of the threaded body 122 has a lead of 0.3 mm and the toothed rack 130 has corresponding teeth. This results in a stroke of the toothed rack 130, and thus of the membrane 100, of 0.3 mm for each full revolution of the threaded body 122.

By lifting the membrane, biomechanical stimuli are applied to the adhering osteoblasts, resulting in osteogenesis. Using the perforations 15 in the membrane, the coagel and the bone callus having formed therein are provided with sufficient circulation via the tissue 165 located over the membrane. Because of the arching of the membrane 100, the bone defect of the jaw bone is thus filled with new bone substance, so that the filled-in bone defect has the convexly shaped arching of the jaw bone. The section of the toothed rack 130 that moves out of the gear system due to the rotation of the threaded body and thus 120 extends into the oral cavity can be removed in a simple and advantageous manner, for example by sawing or milling it off. The distraction device will then not protrude over the upper edge of the teeth in an interfering manner.

1. A distraction device for bone regeneration, comprising a distraction membrane and an actuating element actuating the distraction membrane, wherein the actuating element comprises a toothed rack.

2. The distraction device according to claim 1, wherein the toothed rack is curved.

3. The distraction device according to claim 1, wherein the distraction membrane is secured to one end of the toothed rack.

4. The distraction device according to claim 1, wherein the distraction membrane is arched at least in one direction.

5. The distraction device according to claim 1,
wherein the distraction device further comprises a gear system for moving the toothed rack, and
wherein the gear system is shaped and sized so that it can be disposed in a stationary manner in a region of the bone to be regenerated.

6. The distraction device according to claim 5,
wherein the gear system comprises a housing having a passage, and a threaded body,
wherein the toothed rack is inserted through the passage substantially without play, and
wherein the threaded body is rotatably mounted in the housing so that the threaded body and the toothed rack are operatively engaged with each other.

7. The distraction device according to claim 5, wherein the gear system can be disposed in a stationary manner in the region of the bone to be regenerated by way of the toothed rack.

8. A method for callus distraction in a jaw region, comprising using the distraction device of claim 1, in an oral cavity.

9. A method for hone regeneration by moray of distraction in a jaw region, comprising using the distraction device of claim 1, in an oral cavity.

10. A kit, comprising a medical membrane for bone regeneration, a toothed rack, a clasp, and a threaded body, wherein the toothed rack can be inserted into the clasp and the threaded body can be mounted in the clasp so that the thread of the threaded body engages the teeth of the toothed rack.

11. The distraction device according to claim 5, wherein the gear system is self-locking.

12. The distraction device according to claim 5, wherein the gear system is shaped and sized so that it can be disposed in a stationary manner in an oral cavity.

13. The distraction device according to claim 11, wherein the gear system is shaped and sized so that it can be disposed in a stationary manner in an oral cavity.

14. The distraction device according to claim 6, wherein the gear system is shaped and sized so that it can be disposed in a stationary manner in an oral cavity by way of the toothed rack.

15. The distraction device according to claim 10, wherein the clasp is a housing.
